(19) United States
(12) Patent Application Publication
(10) Pub. No.: US 2021/0236121 A1
(43) Pub. Date: Aug. 5, 2021

(54) SURGICAL NEEDLE SAFETY SHIELD AND CONTAINER FOR HOLDING THE SAME

(71) Applicant: XERICON MEDICAL INC., Dartmouth (CA)

(72) Inventors: Ron O'NEILL, Clearwater, FL (US); Scott FARRELL, Dartmouth (CA)

(21) Appl. No.: 17/048,996

(22) PCT Filed: Apr. 18, 2019
(86) PCT No.: PCT/CA2019/050486

§ 371 Date: Oct. 19, 2020

(30) Foreign Application Priority Data
Apr. 19, 2018 (CA) 3002180

(51) Int. Cl. A61B 17/06; A61B 50/33

(52) U.S. Cl. CPC A61B 17/06114; A61B 50/33; A61B 2017/0608; A61B 2017/06142; A61B 17/06161

(57) ABSTRACT

Disclosed is a surgical needle safety shield for retaining surgical needles. The safety shield having a housing that can be split to allow for user-initiated placement and removal of the surgical needle therefrom. The safety shield also having a needle access port within the housing to allow for a portion of the surgical needle to be grasped to facilitate removal and insertion of the surgical needle in the housing. Also disclosed is a container for storing one or more surgical needle safety shields and a kit made up of such a container and one or more surgical needle safety shields.

### FIELD OF THE INVENTION

The present invention generally relates to a medical device. In particular, the present invention relates to a surgical needle safety shield and container for holding the same.

### BACKGROUND OF THE INVENTION

The design and packaging of surgical needles that are attached to sutures has not changed significantly in recent years. A safety concern with the use of surgical needles is the risk of needle stick injury. Surgical needles are often kept on the surgical field unshielded and held only with a hemostat or similar instrument leaving the needle tip unprotected. Such handling is necessary for the performance of certain surgical procedures but it increases the risk of injury to the surgeon, operating room nurses and the patient.

In addition, surgical needle loss during a procedure is a common occurrence. During normal use, the surgical suture package containing the surgical needle is normally opened and discarded. The package may not be used by the surgical team to reconcile their needle count. As such, there is no reliable way to determine if the number of packages opened and needles used matches the number of surgical needles accounted for at the end of the procedure.

Therefore, there is a need for a shield to protect the suture needle when it is not in use, both on the surgical field and on the instrument table, in order to increase the safety of the surgical team by reducing needle stick injuries and to assist with the surgical needle count at the conclusion of the surgical procedure.

### SUMMARY OF THE INVENTION

According to an aspect of the present invention, there is provided a surgical needle safety shield having a housing for retaining a surgical needle therein, at least a portion of the housing being split to allow for user-initiated placement and removal of the surgical needle therefrom; and a needle access port within the housing to allow for a portion of the surgical needle to be grasped to facilitate removal and insertion of the surgical needle in the housing. The housing surrounds at least a patient interfacing end of the surgical needle.

In one embodiment, at least a portion of the housing is sectioned to form a tab within the housing, and the tab is urged against the housing to retain the surgical needle therein. In a further embodiment, the tab comprises two wings and each wing is urged against the housing to retain the surgical needle therein.

In another embodiment, the housing is split in two segments along a longitudinal axis and the two segments are spring-biased towards one another.

In a further embodiment, stops are provided within the housing to prevent uninitiated removal of the surgical needle from the housing. In another embodiment, the stops are provided in connection with the tabs to prevent uninitiated removal of the surgical needle from the housing. In a yet further embodiment, at least two stops are provided, the first stop being positioned near a patient interfacing end of the surgical needle and the second stop being positioned near the eyelet end of the surgical needle. In a still further embodiment, the stops have two mated portions to permit user-initiated passage of the surgical needle through the stops. In yet another embodiment, the stops are associated with ramps for funneling the surgical needle into the housing for retention therein.

In a still further embodiment, the surgical safety shield is provided with a groove within the housing that corresponds to the shape of the surgical needle being retained therein. In another embodiment, the groove is provided on the tab within the housing.

In one embodiment, the perimeter of the housing has one or more slits to allow passage of a suture thread attached to the surgical needle.

In another embodiment, the housing is U-shaped, ring-shaped, or has a semi-rounded body.

In yet another embodiment, grips are provided on the housing to facilitate handling of the shield.

In a further embodiment, the surgical needle safety shield further comprises a channel around the housing to guide a suture thread attached to the surgical needle.

In a still further embodiment, the needle access port allows access to the eyelet end of the surgical needle.

In another embodiment, the surgical needle safety shield described above further comprises lips positioned at the entrance of the housing to assist in the user-initiated placement of the surgical needle in the housing.

According to another aspect of the present invention, there is provided a container for storing one or more of the surgical needle safety shields described above. The container having a tray; and one or more holders interconnected with the tray and dimensioned to receive the surgical needle safety shield described above.

In one embodiment, the one or more holders are provided in two rows on the tray.

In a further embodiment, the container further comprises a connector positioned on the tray for connecting a second tray. In another still further embodiment, the connector is a hinge to allow for one of the trays to be folded onto the other tray.

According to an aspect of the present invention, there is provided a kit comprising a plurality of surgical needle safety shields described above and the container described above.

In one embodiment, the one or more holders are provided in two rows on the tray.

In a further embodiment, the kit further comprises a connector positioned on the tray for connecting a second tray. In a still further embodiment, the connector is a hinge to allow for one of the trays to be folded onto the other tray.

### BRIEF DESCRIPTION OF THE DRAWINGS

These and other features, aspects and advantages of the present invention will become better understood with regard to the following description and accompanying drawings wherein:

FIG. 1 depicts a surgical needle safety shield according to an embodiment of the present invention;

FIG. 2 shows one side of the surgical needle safety shield shown in FIG. 1;

FIG. 3 shows a cross-sectional view of the surgical needle safety shield shown in FIGS. 1 and 2;

FIG. 4 depicts a surgical needle safety shield according to an embodiment of the present invention;

FIG. 5 shows a cross-sectional view of the surgical needle safety shield shown in FIG. 4;

FIG. 6 depicts a surgical needle safety shield according to an embodiment of the present invention;

FIG. 7 depicts a surgical needle safety shield according to an embodiment of the present invention;

FIG. 8 shows a cross-section view of the surgical needle safety shield shown in FIG. 5;

FIG. 9 shows one-side of the surgical needle safety shield shown in FIG. 5 having a backstop;

FIG. 10 shows a cross-sectional view of the surgical safety shield shown in FIG. 7;

FIG. 11 depicts a surgical needle safety shield according to an embodiment of the present invention;

FIG. 12 shows a cross-sectional view of the surgical safety shield shown in FIG. 9;

FIG. 13A-C depicts a surgical needle safety shield according to an embodiment of the present invention; A) shows a side elevational view of the shield; B) shows a front elevational view of the shield; and C) shows the shield in an open position;

FIG. 14A and B represent a surgical needle shield in operation;

FIG. 15 represents a container for holding surgical needle shields according to an embodiment of the present invention; and

FIG. 16 shows a container for holding surgical needle shields according to an embodiment of the present invention

### DETAILED DESCRIPTION

The following description is of an illustrative embodiment by way of example only and without limitation to the combination of features necessary for carrying the invention into effect.

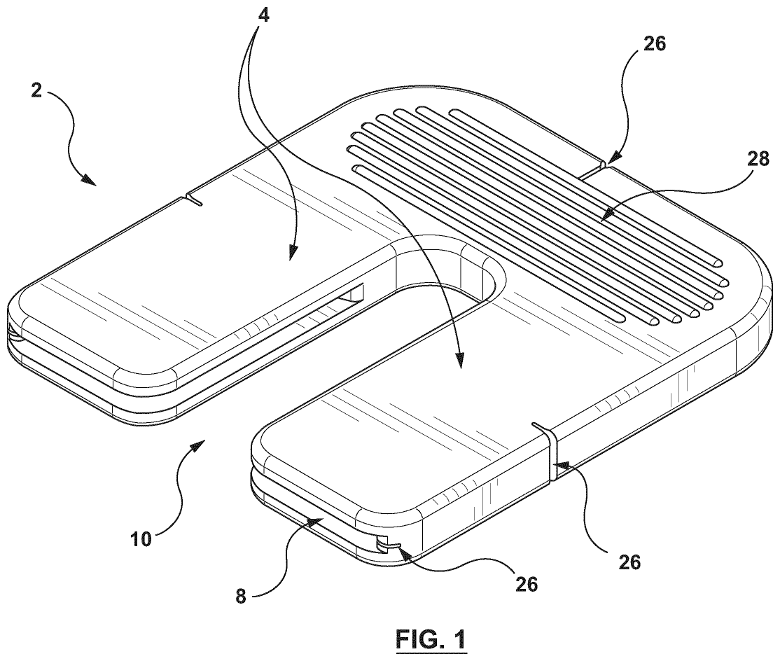

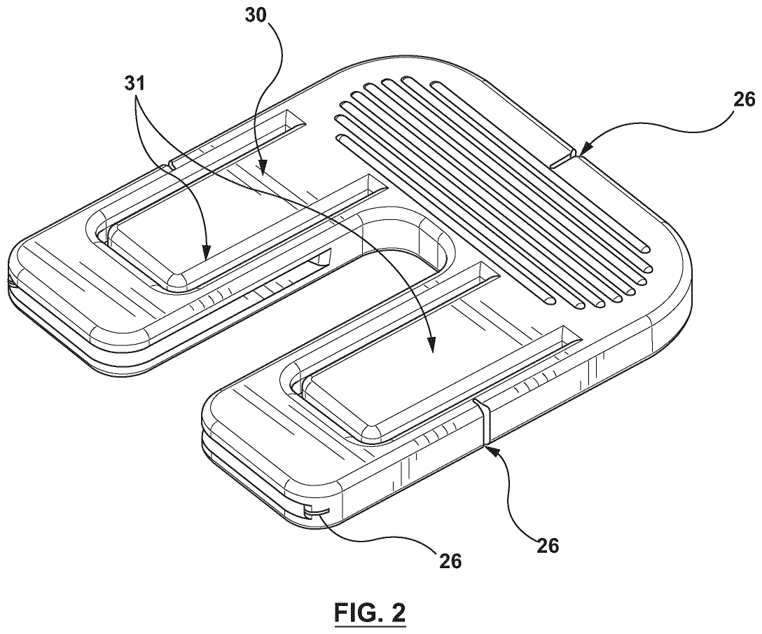

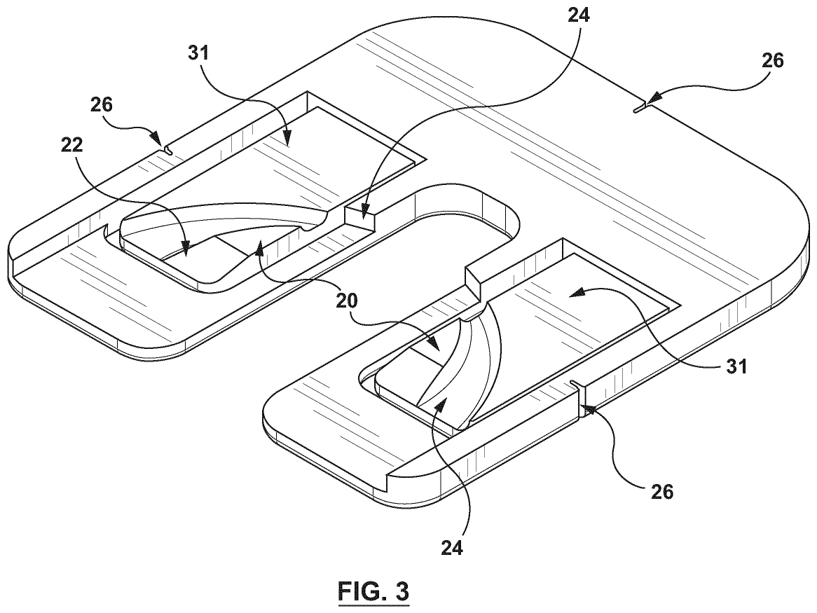

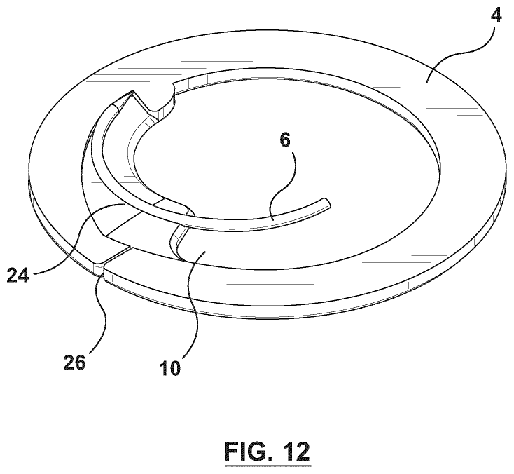

According to an aspect of the present invention and with reference to embodiments presented in FIGS. 1 to 16, there is provided a surgical needle safety shield 2. The surgical needle safety shield 2 having a housing 4 for retaining a surgical needle 6 therein, and a needle access port 10 within the housing 4. At least a portion of the housing 4 being split to allow for user-initiated placement and removal of the surgical needle 6 therefrom. The needle access port 10 allows for a portion of the surgical needle 6 to be grasped to facilitate removal and placement of the surgical needle 6 from the housing 4. The surgical needle safety shield 2 permits the user to shield the surgical needle 6 to prevent needle stick injury by having the patient interfacing end of the surgical needle 6 surrounded by the housing 4, when the surgical needle 6 is in the housing 4.

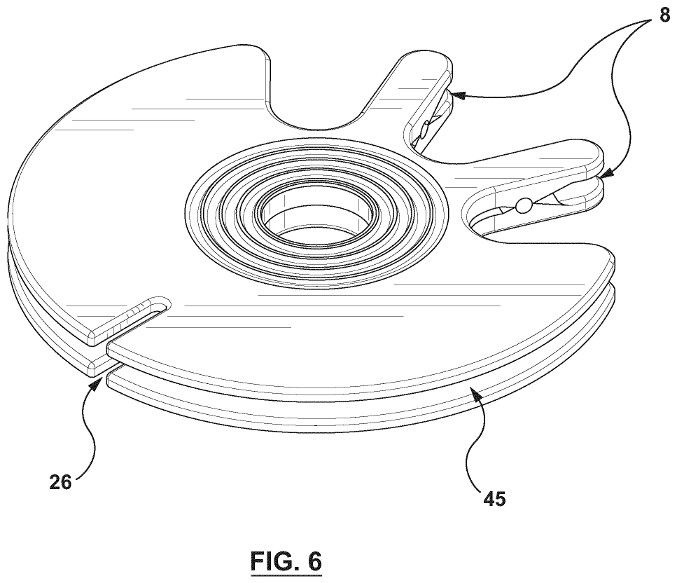

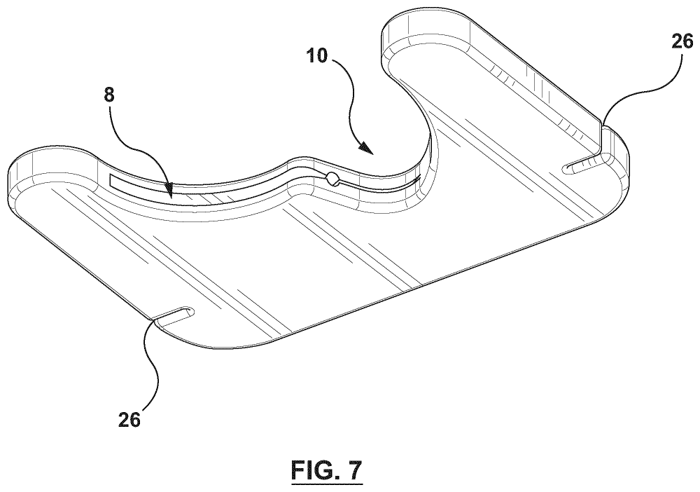

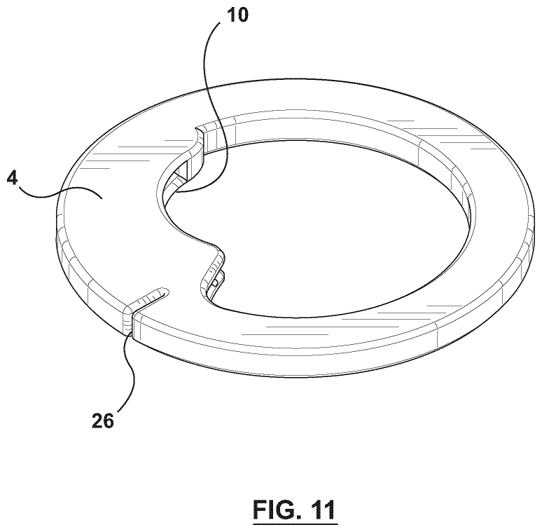

FIG. 1 depicts a surgical needle safety shield 2 where the housing 4 is generally U-shaped. However, as shown in FIGS. 6, 7, 11 and 13, the surgical needle safety shield 2 can be provided with a housing 4 of various shapes and sizes. For example, FIG. 6 shows a surgical needle safety shield 2 where the housing 4 is generally circular; FIG. 7 depicts a surgical needle safety shield 2 where the housing 4 is generally rectangular; and FIG. 11 shows a surgical needle safety shield 2 where the housing 4 is generally ring-shaped. In each of these embodiments, the housing 4 is split to allow for user-initiated placement and removal of the surgical needle 6.

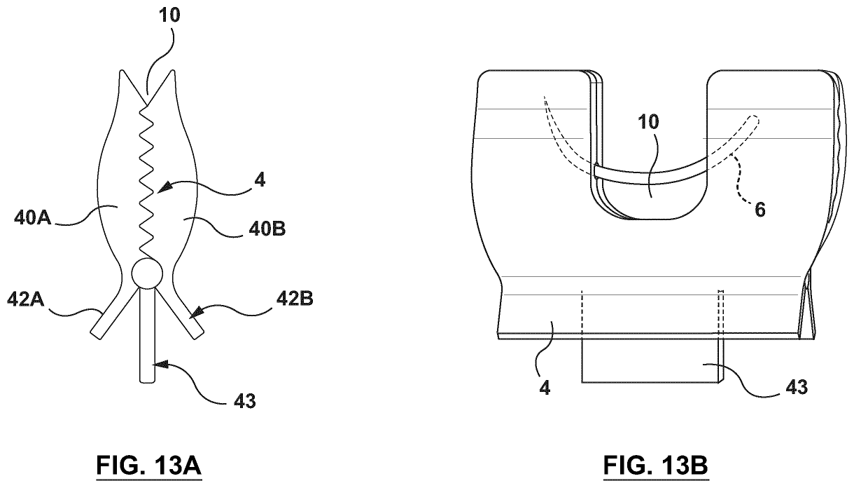

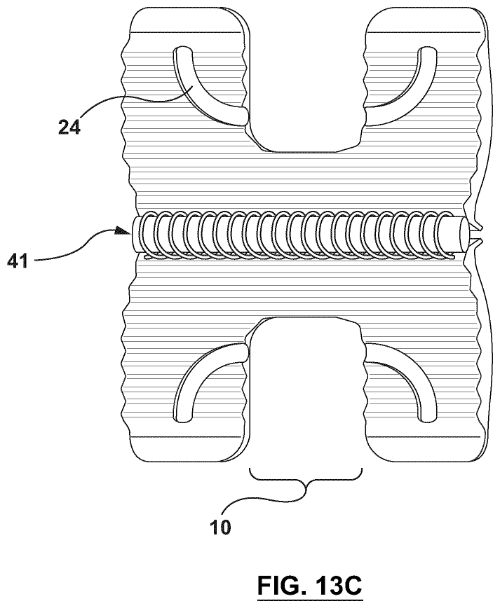

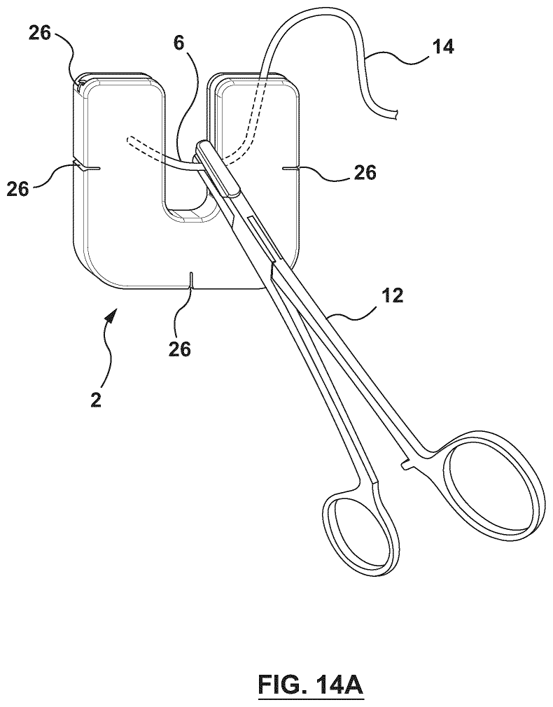

The splitting of the housing 4 creates a passage for the surgical needle 6 to be placed in, and removed from, the housing 4. As shown in FIG. 13A and B, the housing 4 can be split in two segments 40A, 40B along a longitudinal axis. In this case, the two segments 40A, 40B can be spring-biased 41 towards one another to retain the surgical needle 6 within the housing 4. However, in an alternate embodiment, shown for example in FIGS. 1, 6, 7 and 11, the housing 4 is not split into two distinct segments that move independent from one another, but instead is split to form a passageway 8 to allow the surgical needle 6 to be inserted and removed from the housing 4.

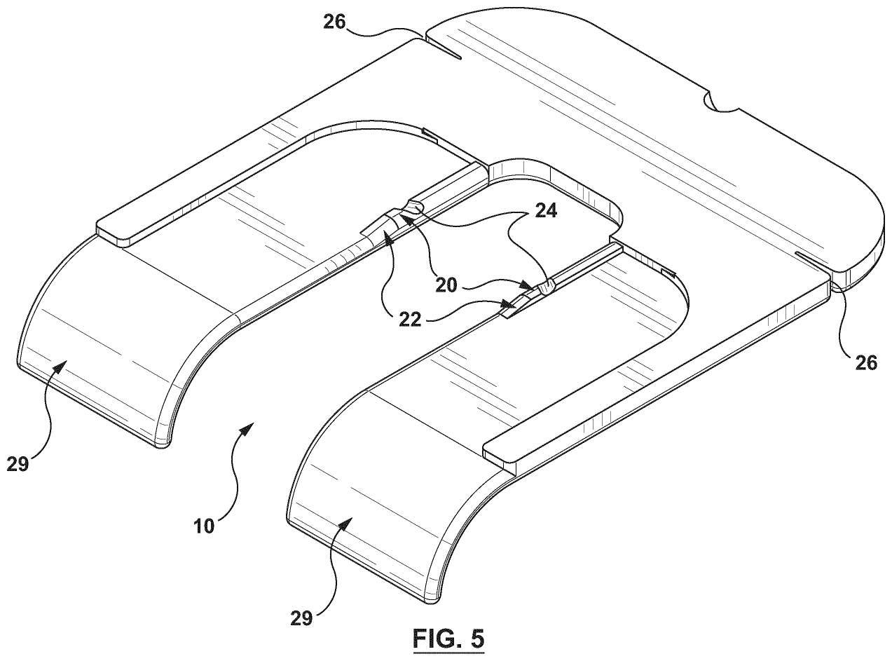

As shown, for example, in FIG. 3, the interior of the housing 4 is provided with a groove 24 that retains the surgical needle 6 within the housing 4 when not in use. The shape and size of the groove 24 generally corresponds to that of the surgical needle 6. As shown in FIG. 5, groove 24 can simply be an indent in the stops 20 that is roughly the diameter of the surgical needle 6. In order to prevent the surgical needle 6 from accidentally sliding out of the groove 24, stops 20 are positioned in the passageway 8 near the groove 24. In most cases, one stop 20 will be positioned near a patient interfacing end of the surgical needle 6 and another stop 20 will be positioned near the eyelet end of the surgical needle 6. The stops 20 provide a physical barrier to prevent the surgical needle 6 from passing from the groove 24 to outside the housing 4 and to limit how far the surgical needle 6 can be inserted into the housing 4. In one embodiment, the stops 20 simply abut the opposite inner wall of the passageway 8 so that the surgical needle 6 can be slid over the stops 20 to insert and remove the needle 6 from the groove 24. In other embodiments, stops 20 can be provided on opposite inner walls of the passageway 8 and configured to mate with one another to allow the surgical needle 6 to slide in between the two stops 20 (see, for example, FIG. 6). To assist in the insertion of the surgical needle 6, ramps 22 can be connected to the stops 20 to funnel the surgical needle 6 into the groove 24. Alternatively, the stops 20 can be provided as wedges where the angled portion of the wedge guides the surgical needle 6 into the groove 24. A second set of ramps or wedges can also be positioned between the stops 20 and the groove 24 to make it easier for the surgical needle 6 to be removed from the groove 24.

As shown in FIGS. 1, 4, 6, 7 and 11, the surgical needle 6 is positioned within the housing 4, by forcibly moving the needle 6 over the stops 20 and into the groove 24. Since the stops 20 must be able to be temporarily separated from either the opposite inner wall of the passageway 8 or a corresponding stop 20, the surgical needle safety shield 2 should be made from a material that has at least some flexibility, such as medical grade silicones, thermoplastic elastomers or polymers; or should be provided with some feature that allows for the stops 20 to be temporarily separated from either the opposite inner wall of the passageway 8 or a corresponding stop 20 to allow for insertion and removal of the surgical needle. In one embodiment, the housing 4 is provided with a tab 30 that is capable of flexing independent of the rest of the housing 4 to allow the surgical needle 6 to be inserted and removed from the housing 4. This particular arrangement allows for the surgical needle safety shield 2 to be made from harder, and potentially cheaper, medical grade polymers, silicones or thermoplastic elastomers thus allowing the surgical needle safety shield 2 to be disposable.

As shown in FIG. 3, when the housing 4 is provided in a generally U-shaped design, the tab 30 can be divided into two separate wings 31 that are positioned in each of the parallel arms of the U-shaped housing 4. When viewed in cross-section, as is shown in FIG. 3, the wings 31 can form a portion of one of the inner walls of the passageway. In the embodiment shown in FIG. 3, the stops 20 and ramps 22 are provided on the wings 31. In operation, the surgical needle 6 is funnelled up the ramps 22 and over the stops 20, which causes the wings 31 to flex away from the rest of the housing 4. Once the surgical needle 6 is forced over the stops 20, the needle 6 will drop into the groove 24 causing the wings 31 to return to their starting position.

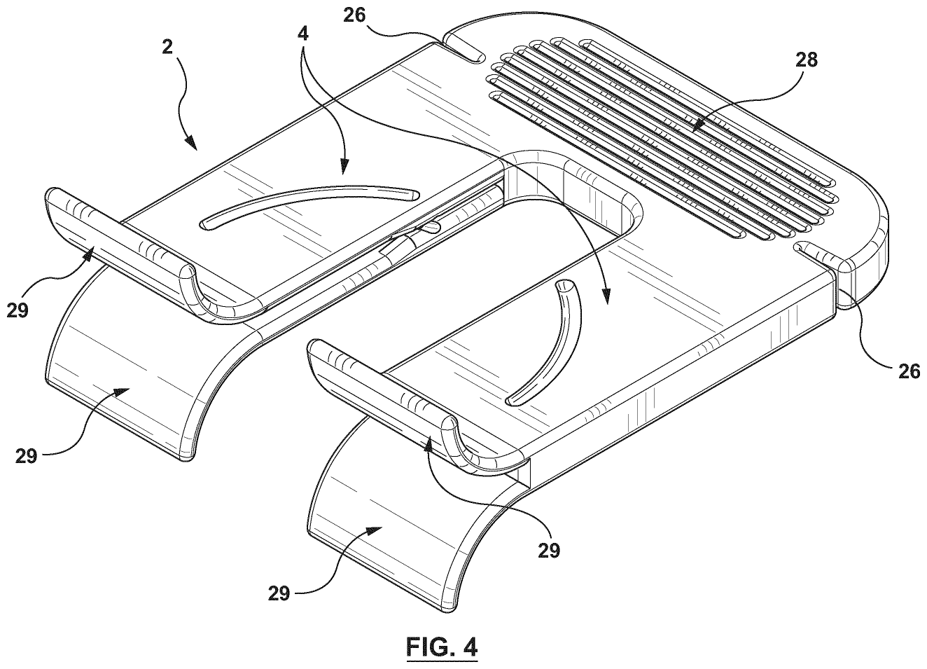

As shown in FIGS. 4 and 5, the housing 2 can be provided with lips 29 at the entrance of the passageway 8. The lips 29 are flared away from the entrance of the passageway 8 of the housing 4 to aid in the insertion of the surgical needle 6 and to permit easier handling and manipulation of the surgical needle safety shield 2, especially when the surgical needle safety shield 2 is being stored or inserted into the container 36 described below.

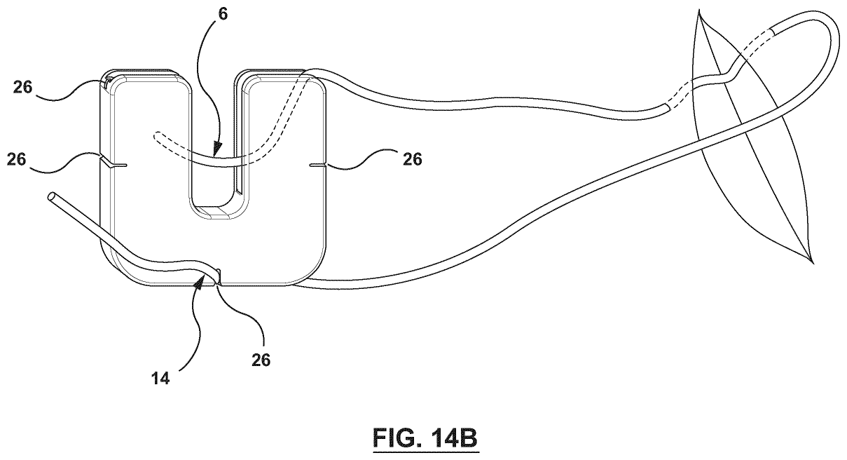

In operation, the surgical needle 6 is inserted into the housing 4 by forcibly moving the needle 6 over the stops 20 into the groove 24. To remove the surgical needle 6 from the groove 24, the reverse process occurs and the needle 6 is forcibly removed from the groove 24 over the stops 20 to outside the housing. The surgical needle 6 is typically inserted and removed from the housing 4 with the use of a suture needle holder 12 or surgical needle driver (see FIG. 14A). In order for the suture needle holder 12 to be able to grasp the surgical needle 6 while it is being inserted and removed from the housing 4, a needle access port 10 is provided within the housing. The needle access port 10 can be provided as a cutout in the housing 4, as shown in FIG. 7, or the housing 4 can be formed in a manner that allows the surgical needle 6 to be grasped while the needle 6 is being inserted or removed from the housing 4 (FIG. 1).

In order to facilitate the handling of the surgical needle safety shield 2, grips 28 may be provided on the housing 4. In most cases, the grips 28 will either be in the form of raised geometric shapes, or, textural differences between the main portion of the housing 4 and the grips 28.

The surgical needle shield 2 can also be provided with one or more slits 26 positioned around the perimeter of the housing 4. The slits 26 allow for suture thread 14 attached to the surgical needle 6 to be retained while the needle 6 is on the surgical field. The slits 26 are dimensioned such that the suture thread 14 can travel through the slit 26, but are narrow enough to prevent the thread 14 from easily falling out of the slit 26. As shown in FIG. 14B, the slits 26 can also be used to safeguard against a surgical needle 6 being removed from a surgical needle shield 2 but not being returned.

As shown in the embodiment of FIG. 6, in some applications, the surgical needle safety shield 2 can be provided with an essentially circular housing 4. In this embodiment, the circumference, at least a portion thereof, can be provided with a channel 45 that can accommodate the suture thread 14 attached to the surgical needle 6. The channel 45 allows for the suture thread 14 to be wound, or partially wound, around the housing 4.

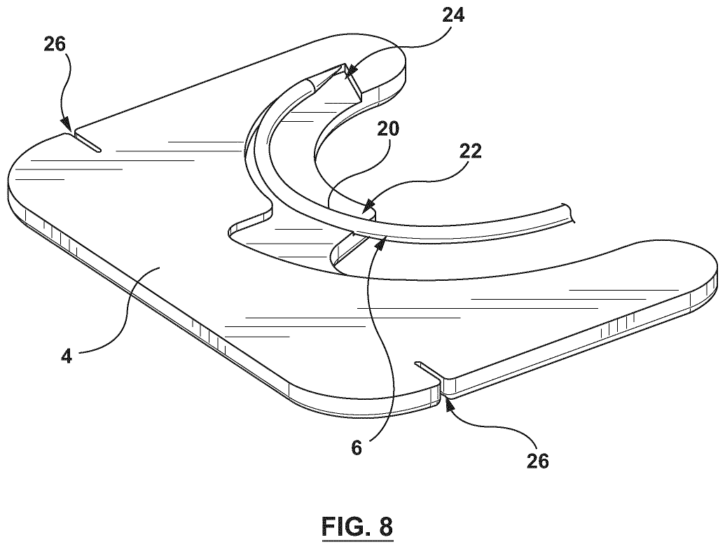

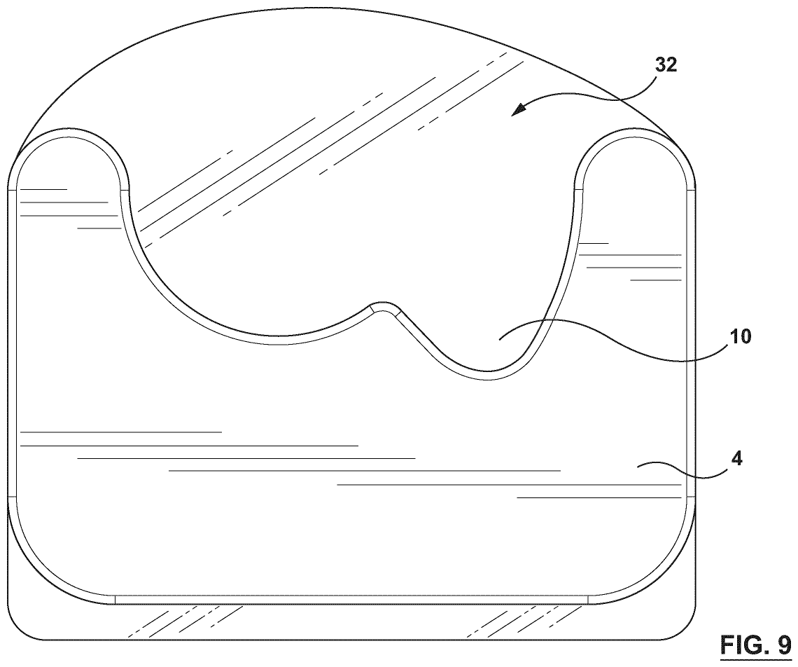

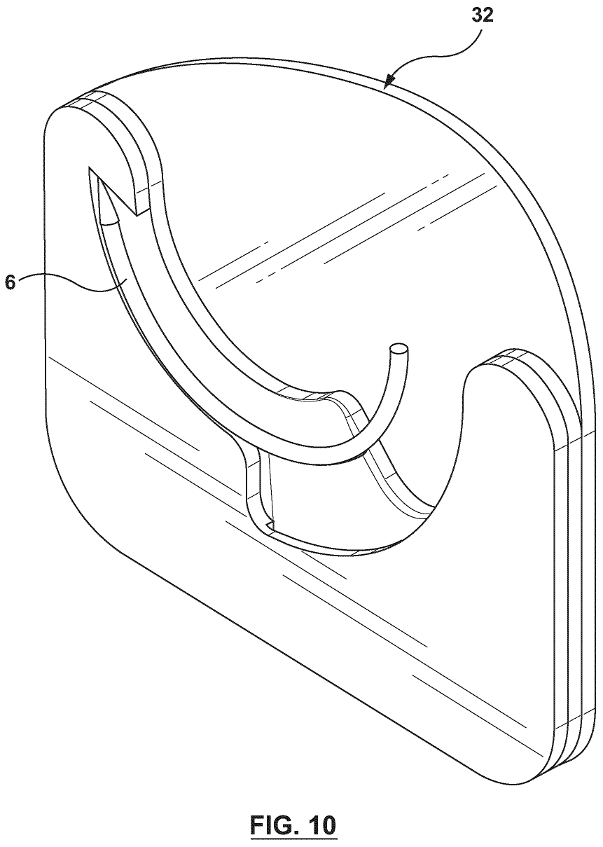

In another embodiment, the housing 4 can be provided so that the surgical needle 6 is first snapped into the groove 24, by forcibly moving the surgical needle 6 over the stops 20 and then rotating the patient interfacing end of the needle 6 towards a blind end (FIG. 8). This arrangement allows for the eyelet end of the surgical needle 6 to be free and capable of manipulation by the user. In an alternate embodiment, the surgical needle safety shield 2 depicted in FIG. 7 can be provided with a backstop 32 that expands one side of the housing 4 (see FIGS. 9 and 10). The backstop 32 provides an additional layer of protection against accidental needle sticks as the user inserts and removes surgical needles 6 from the surgical needle safety shield 2.

In order to provide the user with a surgical needle safety shield 2 that is easier to handle, the housing 4 may be provided in the form of a ring (FIG. 11). The non-needle engaging portion of the housing 4 can make the shield 2 easier to remove and insert into a container, as described below.

As shown in FIGS. 13A and B, the housing 4 of the surgical needle safety shield 2 can be split into two segments 40A and 40B. The two segments 40A and 40B are connected through a spring mechanism 41. The spring mechanism 41 biases the two segments 40A and 40B towards one another. In operation, the base sections 42A and 42B of the two segments 40A and 40B are squeezed together causing the spring-biased force to be overcome, which results in the housing 4 opening to receive the surgical needle 4 in the groove 24. In this arrangement, the housing 4 can further comprise a tab 43 that permits the surgical needle safety shield 2 to be inserted into a container, as described below.

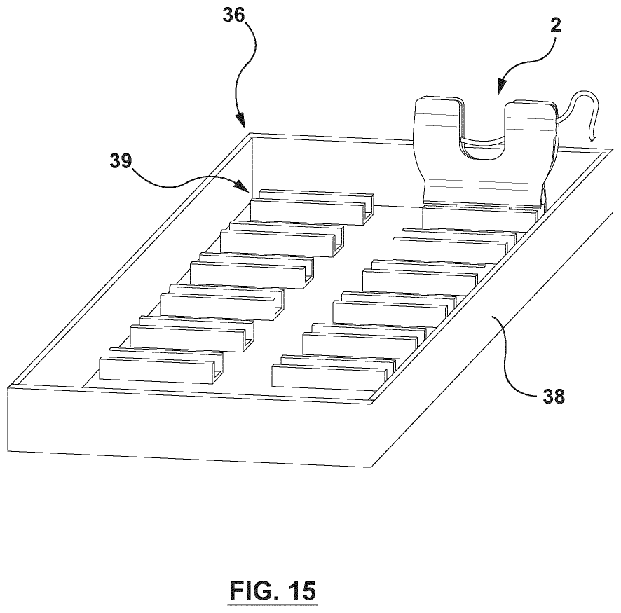

According to a further aspect of the present invention and referring to FIG. 15, there is provided a container 36 for storing one or more of the surgical needle safety shields 2. The container 36 is made up of a tray 38 and one or more holders 39 connected to the tray 38. The holders 39 are dimensioned to receive the surgical needle safety shields 2 and can be of different size and shape depending upon the size and configuration of the surgical needle safety shield 2. In most cases, the one or more holders 39 are provided in rows within the tray 38. When at least two rows are provided, loaded (i.e. containing a surgical needle 6) surgical needle safety shields 2 are inserted into one row and then transferred to the second row, once the surgical needle 6 has been used and safely returned to the surgical needle safety shield 2 from which it originated. This arrangement allows for reliable accountability of needles used and returned, thus minimizing the possibility that a surgical needle is left in the operating field.

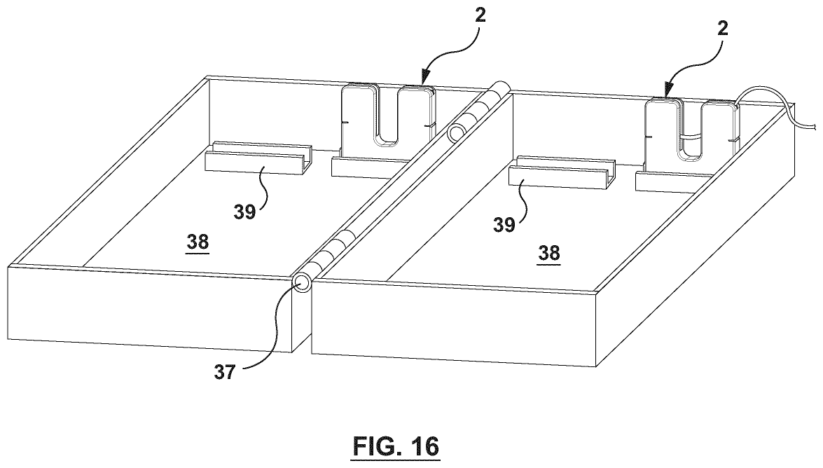

According to a further aspect of the present invention and referring to FIG. 16, during complex procedures, where many surgical needles 6 are needed, it is possible to link more than one container to form a loaded surgical needle safety shield 2 tray 38 and a used surgical needle safety shield 2 tray 38. In this case, instead of transferring the surgical needle safety shields 2 from one row to another within the same tray, the surgical needle safety shields 2 are transferred from one tray 38 to the other tray 38. To save space, the two trays 38 may be linked through a hinge 37, which allows for the trays 38 to be folded onto one another.

According to a further aspect of the present invention, there is provided a kit comprising a plurality of surgical needle safety shields 2 and the container 36 (FIG. 15, 16). As described above, the kit may comprise one or more holders 39 in two rows on a tray 38 and/or a second tray 38.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein set forth, and as follows in the scope of the appended claims.

1. A surgical needle safety shield comprising:
a housing for retaining a surgical needle therein, at least a portion of the housing being split to form a passageway to allow for user-initiated placement and removal of the surgical needle from the housing;
one or more bidirectional stops positioned in the passageway to prevent the surgical needle retained in the housing from moving out of the passageway without the user engaging the surgical needle; and
a needle access port within the housing to allow for a portion of the surgical needle to be grasped to facilitate removal and insertion of the surgical needle through the passageway and over the one or more bidirectional stops,
wherein the housing surrounds at least a patient interfacing end of the surgical needle, and
wherein resistance provided by the one or more bidirectional stops against the user-initiated placement of the surgical needle into the housing is substantially equal to the resistance provided by the one or more bidirectional stops against the user-initiated removal of the surgical needle from the housing.

2. The surgical needle safety shield according to claim 1, wherein at least a portion of the housing is sectioned to form a tab within the housing, and wherein the tab is urged against the housing to retain the surgical needle therein.

3. The surgical needle safety shield according to claim 2, wherein the tab comprises two wings and each wing is urged against the housing to retain the surgical needle therein.

4-6. (canceled)

7. The surgical needle safety shield according to claim 1, wherein at least two bidirectional stops are provided, the first stop being positioned near the patient interfacing end of the surgical needle and the second stop being positioned near the eyelet end of the surgical needle.

8. The surgical needle safety shield according to claim 1, wherein the one or more bidirectional stops have two mated portions to permit user-initiated passage of the surgical needle through the stops.

9. The surgical needle safety shield according to claim 1, wherein the one or more bidirectional stops are associated with ramps for funneling the surgical needle into the housing for retention therein.

10. The surgical needle safety shield according to claim 1, further comprising a groove within the housing that corresponds to the shape of the surgical needle being retained therein.

11. (canceled)

12. The surgical needle safety shield according to claim 1, wherein the perimeter of the housing has one or more slits to allow passage of a suture thread attached to the surgical needle.

13. The surgical needle safety shield according to claim 1, wherein the housing is U-shaped, ring shaped or has a semi-rounded body.

14. The surgical needle safety shield according to claim 1, wherein grips are provided on the housing to facilitate handling of the shield.

15. (canceled)

16. (canceled)

17. The surgical needle safety shield according to claim 13, further comprising a channel around the housing to guide a suture thread attached to the surgical needle.

18. The surgical needle safety shield according to claim 1, wherein the needle access port allows access to the eyelet end of the surgical needle.

19. The surgical needle safety shield according to claim 1, further comprising lips positioned at the entrance of the housing to assist in the user-initiated placement of the surgical needle in the housing.

20. A container for storing one or more of the surgical needle safety shields of claim 1, the container comprising:
two trays; and
one or more holders interconnected with the tray and dimensioned to receive the surgical needle safety shields.

21. The container of claim 20, wherein the one or more holders are provided in two rows on the tray.

22. The container of claim 20, further comprising a connector positioned on the tray for connecting a second tray.

23. The container of claim 22, wherein the connector is a hinge to allow for one of the trays to be folded onto the other tray.

24-27. (canceled)

28. The container of claim 20, wherein the one or more holders in one of the trays are dimensioned to hold the surgical needle safety shields in an upright position and the one or more holders in the other tray are dimensioned to hold the surgical needle safety shields in an inverted position.

29. A method of using the surgical needle safety shields of claim 1 and the container of claim 20 in an operating environment, the method comprising the steps of:
providing the surgical needle safety shield with a surgical needle contained therein;
removing the surgical needle from the surgical needle safety shield for use in the operating environment;
placing the empty surgical needle safety shield in one of the holders of one of the two trays of the container;
returning the surgical needle to the empty surgical needle safety shield after use in the operating environment; and
placing the surgical needle safety shield containing the surgical needle in one of the holders of the other tray of the container.

30. The method of claim 29, wherein the empty surgical needle safety shield is placed in one of the holders of one of the two trays of the container in an position where the needle access port faces away from the holder and the surgical needle safety shield containing the surgical needle is placed in one of the holders of the other tray of the container in a position where the needle access port faces towards the holder.
